(19) United States
(12) Patent Application Publication
(10) Pub. No.: US 2018/0092519 A1
(43) Pub. Date: Apr. 5, 2018

(54) INFRARED FLUORESCENCE OBSERVATION DEVICE

(71) Applicant: OLYMPUS CORPORATION, Tokyo (JP)

(72) Inventor: Shuichi Kato, Tokyo (JP)

(73) Assignee: OLYMPUS CORPORATION, Tokyo (JP)

(21) Appl. No.: 15/821,016

(22) Filed: Nov. 22, 2017

Related U.S. Application Data
(63) Continuation of application No. PCT/JP2015/065750, filed on Jun. 1, 2015.

(51) Int. Cl. A61B 1/04; A61B 1/06; A61B 1/00; G01N 21/64

(52) U.S. Cl. CPC A61B 1/043; A61B 1/0638; G01N 21/64; A61B 1/00009; A61B 1/0661

(57) ABSTRACT

An infrared fluorescence observation device includes a light source configured to irradiate visible light and excitation light including a longer wavelength band than the visible light, an imaging unit on which generates a first image on the basis of second light obtained by attenuating a wavelength band including the excitation light from first light emitted from the subject irradiated with at least the excitation light and generates a second image on the basis of third light obtained by eliminating only the wavelength band of the fluorescence including a longer wavelength band than the excitation light from the second light, and a signal processing unit generates a third image according to light of a wavelength band including the fluorescence using the first image and the second image.

### TECHNICAL FIELD

The present invention relates to an infrared fluorescence observation device.

The present application is a continuation application based on PCT Patent Application No. PCT/JP2015/065750, filed on Jun. 1, 2015.

### BACKGROUND ART

Conventionally, a diagnostic method of determining the presence or absence of a lesion by administering a fluorescent drug called indocyanine green (ICG) into a body of a test subject person in advance in order to diagnose cancer or the like is known. ICG is a fluorescent substance having affinity for a lesion such as cancer and is excited by light in an infrared region and emits fluorescence. Various medical systems having a function of observing the fluorescence emission of ICG have been conventionally proposed. Also, for example, an examiner such as a doctor determines the presence or absence of a lesion from the brightness of the fluorescence emission observed using the medical system.

In a conventional medical system, as a configuration for observing fluorescence emission, an infrared fluorescence observation device, which irradiates light of an infrared region such as near infrared light as excitation light for exciting ICG and images a specific protein in a lesion portion from which fluorescence is emitted due to the irradiated excitation light, is provided.

For example, Japanese Patent No. 3962122 discloses an endoscope device capable of allowing observation of fluorescence using excitation light in addition to normal observation using visible light. In the endoscope device disclosed in Japanese Patent No. 3962122, visible light and excitation light are irradiated from a distal end of art insertion unit to a test subject, and visible light and excitation light reflected from the test subject, and fluorescence emitted by ICG through excitation by the excitation light are guided to a camera head via an image guide fiber. In the endoscope device disclosed in Japanese Patent No. 3962122, the visible light, the excitation light, and the fluorescence guided to the camera head are separated into visible light, excitation light, and fluorescence by a dichroic mirror provided in the camera head. The separated visible light is imaged by an imaging means. Also, in the endoscope device disclosed in Japanese Patent No. 3962122, the excitation light is eliminated (cut) from the separated excitation light and fluorescence by an excitation light cut filter provided in the camera head, and only the fluorescence is, amplified by an image intensifier and imaged by an imaging means different from an imaging means for the visible light.

Also, in another configuration of the endoscope device disclosed in Japanese Patent No. 3962122, only visible light is irradiated to the test subject during normal observation, and only excitation light is irradiated to the test subject during fluorescent photographing. In the endoscope device disclosed in Japanese Patent No. 3962122, the imaging means images the visible light reflected from the test subject during normal observation. Also, in the endoscope device disclosed in Japanese Patent No. 1962122, during the fluorescein photographing only the excitation light is eliminated by the excitation light cut filter from the excitation light and the fluorescence reflected from the test subject and only the fluorescence is imaged by the imaging means which is the same as the imaging means for visible light.

### SUMMARY OF INVENTION

According to the first aspect of the present invention, an infrared fluorescence observation device includes a light source configured to irradiate visible light and excitation light including a longer wavelength band than the visible light; an imaging unit on which the visible light, the excitation light, and fluorescence including a longer wavelength band than the excitation light are incident from a subject irradiated by the light source; and a signal processing, unit configured to process a signal obtained from the imaging unit, wherein the imaging unit includes a first wavelength selection unit configured is input first light from the subject irradiated with at least the excitation light and output second light obtained by attenuating a wavelength band including the excitation light from the first light; a half mirror configured to divide the second light into a first optical path and a second optical path; a first imagine element arranged in the first optical path and configured to generate a first image on the basis of the second light; a second wavelength selection unit arranged in the second optical path, to which the second light is input, and from which third light obtained by eliminating only a wavelength band including the fluorescence from the second light is output; and a second imaging element arranged in the second optical path and configured to generate a second image on the basis of the third light, and wherein the signal processing unit generates a third image according to light of a wavelength band including the fluorescence using the first image and the second image.

According to a second aspect of the present invention, in the infrared fluorescence observation device of the above-described first aspect, the signal processing unit may generate the third image by subtracting the second image from the first image.

According to the third aspect of the present invention, in the infrared fluorescence observation device of the above-described first aspect, the infrared fluorescence observation device may be an endoscope device, the endoscope device may include a scope unit including an insertion unit configured to be inserted into a body and an operation unit configured to operate the insertion unit; and an external processing unit connected to the scope unit, the light source and the imaging unit may be arranged in the scope unit, and the signal processing unit may be arranged in the external processing unit.

According to the fourth aspect of the present invention, in the infrared fluorescence observation device of the above-described first aspect, the infrared fluorescence observation device may be a microscope device.

### BRIEF DESCRIPTION OF DRAWINGS

FIG. 1 is a diagram showing a schematic configuration of an endoscope device according to a first embodiment of the present invention.

FIG. 2A is a diagram schematically showing an example of an operation in the endoscope device of the first embodiment of the present invention.

FIG. 2B is a diagram schematically showing an example of an operation in the endoscope device of the first embodiment of the present invention.

FIG. 3 is a diagram showing a schematic configuration of an endoscope device according to a second embodiment of the present invention.

FIG. 4 is a diagram showing a schematic configuration of an endoscope device according to a third embodiment of the present invention.

FIG. 5 is a diagram showing an example of an arrangement of pixels in an imager provided in the endoscope device of the third embodiment of the present invention.

### DESCRIPTION OF EMBODIMENTS

### First Embodiment

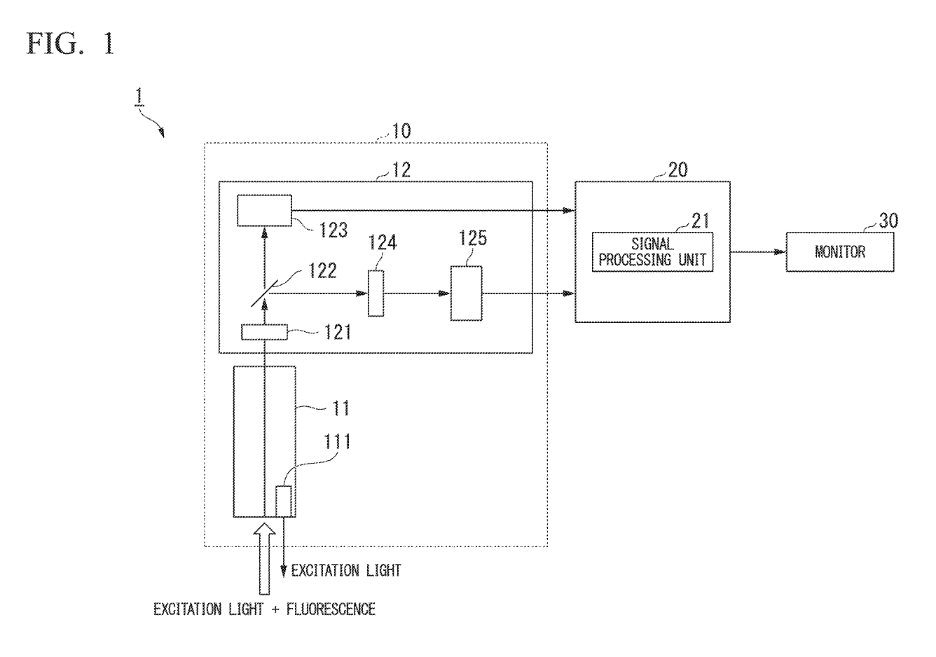

Hereinafter, embodiments of the present invention win be described with reference to the drawings. In the following description, a case in which an infrared fluorescence observation device often present invention is configured as an endoscope device will be described. FIG. 1 is a diagram showing a schematic configuration of an endoscope device according to the first embodiment of the present invention.

An endoscope device 1 of the first embodiment is a rigid endoscope for laparoscopic surgery. The endoscope device 1 is used for a test subject person in a state in which a fluorescent drug such as ICG has been administered into his/her body in advance. In the following description, it is assumed that ICG is administered as the fluorescent drug into the body of the test subject person.

Also, the endoscope device 1 is an endoscope device having a function of photographing a test subject with visible light and a function of photographing the test subject with fluorescence emitted by excitation of administered ICG through irradiation of excitation light such as near infrared light. In the following description, for ease of description, the configuration of the endoscope device 1 for implementing the function of photographing the test subject with fluorescence will be described. Also, for example, the function of photographing the test subject with visible light in the endoscope device 1 can be implemented by photographing the test subject with visible light separated by a dichroic mirror as in a conventional endoscope device. That is, the configuration of the endoscope device 1 to be described below is a configuration in which the function of photographing the test subject with fluorescence is implemented by arranging the infrared fluorescence observation device of the present invention, on an optical path of the excitation light and the fluorescence separated by the dichroic mirror. Also, the configuration of the endoscope device 1 to be described below is also, for example, a configuration in which the function of photographing the test subject is implemented with fluorescence by operating the infrared fluorescence observation device of the present invention during fluorescent photographing in which only excitation light is irradiated to the test subject in the conventional endoscope device.

In FIG. 1, the endoscope device 1 includes a scope unit 10, an external processing unit 20, and a monitor 30. In the endoscope device 1, the scope unit 10 includes an insertion unit 11 and an operation unit 12. In the scope unit 10, the insertion unit 11 includes a light source 111 which is a constituent element of the infrared fluorescence observation device of the present invention. Also, in the scope unit 10, the operation unit 12 is configured to include an imaging tint having, an excitation light cut filter 121, a half mirror an imager 123, a fluorescence cut filter 124, and an imager 125, which are constituent elements of the infrared fluorescence observation device of the present invention. Also, in the endoscope device 1, the external processing unit 20 is configured to include a signal processing unit 21 which is the infrared fluorescence observation device of the present invention.

In the endoscope device 1 according to the first embodiment, the infrared fluorescence observation device of the present invention includes the light source 111, the imaging unit (the excitation light cut filter 121, the half mirror 122, the imager 123, the fluorescence cut filter 124, and the imager 125), and the signal processing unit 21.

The insertion unit 11 provided in the scope unit 10 is inserted into a body of a test subject person in a state in which ICG has been administered in advance. The light source 111 provided in the insertion unit 11 is a light source that emits near infrared light for exciting ICG as excitation light. The light source 111 is arranged at a distal end of the insertion unit 11, and irradiates the emitted excitation light to the test subject. Thereby, the excitation light reflected from the test subject and fluorescence emitted through excitation of ICG by the excitation light is incident on the distal end of the insertion unit 11. The insertion unit 11 guides the incident excitation light and fluorescence to the operation unit 12 provided in the scope unit 10.

Also, in the present invention, a method and configuration for guiding the excitation light and the fluorescence incident on the distal end of the insertion unit 11 to the operation unit 12 are not particularly limited. For example, there may be a so-called relay lens or pupil relay method for transmitting an image of light in a relay form. Also, for example, a configuration with an optical fiber such as an it guide fiber may be used. Also, in the present invention, the light source 111 is arranged at the distal end of the insertion unit 11, but the light source 111 may be arranged or installed at other positions. For example, another device (a light source unit) in which the light source 111 is installed may be provided, and the light emitted by the light source 111 may be guided to the insertion unit 11 through an optical waveguide such as an optical fiber.

Because the endoscope device 1 is an endoscope device for laparoscopic surgery as described above, external light such as visible light is not basically incident. However, it is conceivable that weak external light (such as visible light) may be incident on the distal end of the insertion unit 11 according to an operation environment in which the endoscope device 1 is used. In the endoscope device 1 of the first embodiment, weak external light incident on the distal end of the insertion unit 11 can also be treated as a noise component in the same manner as weak excitation light irradiated to the imager 125. Accordingly, in the following description, it is assumed that visible light is not incident on the endoscope device 1 for ease of description.

The operation unit 12 is a support unit that controls the operation of the insertion unit 11 through an operation of, for example, an examiner (for example, a doctor who is performing laparoscopic surgery or the like). The imaging unit provided in the operation unit 12 outputs each of pixel signals obtained thorough photographing of the imager 123 and the imager 125 to the external processing unit 20.

The excitation light cm filter 121 is an optical filter that reflects or absorbs and attenuates incident excitation light and only excitation light included in the fluorescence. The excitation light cut filter 121 emits light (fluorescence) obtained by attenuating the excitation light to the half mirror 122.

Also, the excitation, light is attenuated by the excitation light cut filter 121 as described above with respect to the excitation light and the fluorescence guided by the insertion unit 11, but it is also conceivable that the excitation light cut filter 121 may not completely attenuate the excitation light. In other words, it is conceivable that the light emitted from the excitation light cut filter 121 may contain a weak excitation light component. In the following description, light emitted from the excitation light cut filter 121 is assumed to contain excitation light which is weak (hereinafter referred to as “weak excitation light”) in addition to fluorescence.

The half mirror 122 is an optical element for splitting (separating) the incident light at 1:1. The half mirror 122 splits the fluorescence and weak excitation light emitted from the excitation light cut filter 121 into an optical path on the imager 123 side and an optical path on the imager 125 side. Thereby, the same light (the fluorescence and the weak excitation light) is incident on the imager 123 and the fluorescence cut filter 124,

The imager 123 is an imaging element that exposes (detects) the incident light and outputs a pixel signal obtained by photoelectrically converting the exposed light. The imager 123 exposes the fluorescence and the weak excitation light emitted from the half mirror 122, and outputs pixel signals corresponding to the fluorescence and the weak excitation light to the external processing unit 20.

The fluorescence cut filter 124 is an optical filter that attenuates only the fluorescence contained in the incident fluorescence and weak excitation light. Because the fluorescence incident on the fluorescence cut filter 124 is significantly weak, the fluorescence component can be attenuated (eliminated) to almost zero by attenuating the fluorescence by the fluorescence cut filter 124. The fluorescence cut filter 124 emits light from which the fluorescence has been eliminated (the weak excitation light) to the imager 125.

The imager 125 is an imaging element similar to the imager 123. Light different from that for the imager 123, that is, the weak excitation light emitted from the fluorescence cut filter 124, is incident on the imager 125. The imager 125 exposes the weak excitation light emitted from the fluorescence cut filter 124, and outputs a pixel signal corresponding to the weak excitation light to the external processing unit 20.

The external processing unit 20 performs predetermined image processing on each pixel signal input from the operation unit 12 provided in the scope unit 101 generate an image of the test subject. The external processing unit 20 outputs the generated image of the test subject to the monitor 30 for display.

The signal processing unit 21 is an image processing device that performs signal processing to be described below on a pixel signal input from the operation unit 12 to generate an image of the test subject. The signal processing unit 21 generates an image according to a pixel signal corresponding to the difference between a pixel signal according to the fluorescence and the weak excitation light input from the imager 123 provided in the operation unit 12 and a pixel signal according to the weak excitation light input from the imager 125 provided in the operation unit 12, that is, an image according to a pixel signal of only a fluorescence component. More specifically, the signal processing unit 21 generates art image according to the pixel signal according to the fluorescence and the weak excitation light input from the imager 123 provided in the operation unit 12, that is, an image of the fluorescence including the weak excitation light. Also, the signal processing unit 21 generates an image according to a pixel signal according to the weak excitation light input from the imager 125 provided in the operation unit 12, that is, an image having only the weak excitation light. Then, the signal processing unit 21 subtracts the image of only the weak excitation light from the image of the fluorescence including the weak excitation light, so that the image formed only with the pixel signal according to the fluorescence, that is, an image of only the fluorescence component, is generated. Here, the subtraction of the image is a difference calculation between the pixels values of the pixels arranged at the same position in each image. Then, the signal processing unit 21, outputs the image of only the generated fluorescence component to the monitor 30.

The monitor 30 is a display device such as, for example, a liquid crystal display (LCD) for displaying the image input from the external processing unit 20.

With such a configuration, the endoscope device 1 excites ICG administered into the test subject person with the excitation light, and presents the image of the test subject according to the fluorescence emitted by the excited ICG to an examiner.

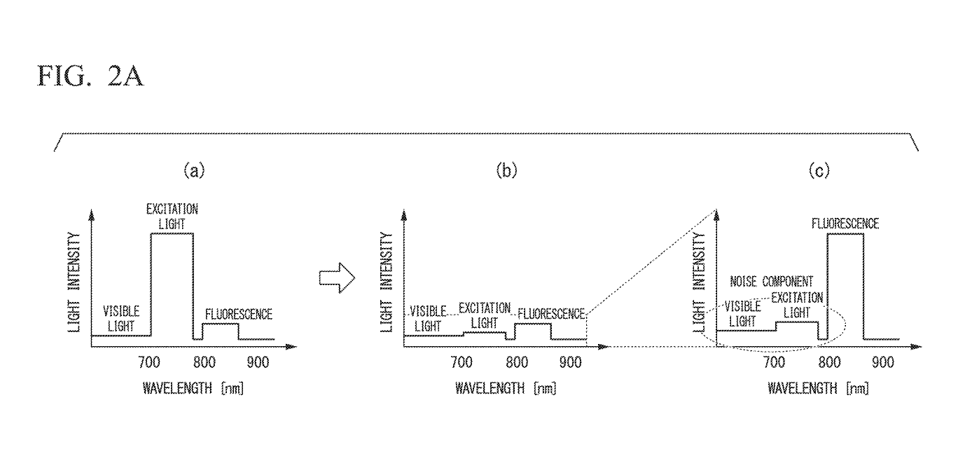

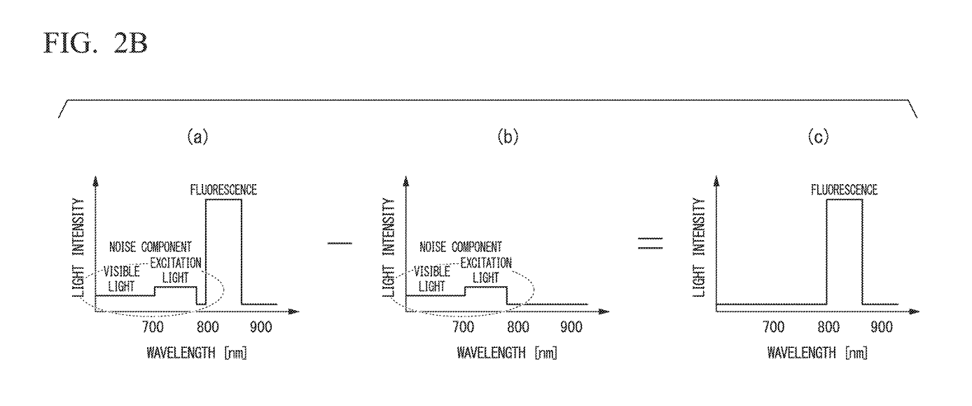

Next, an operation of the endoscope device 1 according to the first embodiment will be described. FIG. 2A and FIG. 2B are diagrams schematically showing an example of an operation in the endoscope device according to first embodiment of the present invention. An operation in which the excitation light cut filter 121 attenuates the excitation light from light incident on the distal end of the insertion unit 11 and guided to the operation unit 12 is schematically shown in FIG. 2A. Also, processing of light after the excitation light is attenuated by the excitation light cut filter 121 is schematically shown in FIG. 2B. In FIG. 2A and FIG. 2B, a wavelength of light is indicated on the horizontal axis and an intensity of light thereinafter referred to as “light intensity”) is indicated on the vertical axis.

Also, a case in which the visible light, which is the weak external light, is incident the distal end of the insertion unit 11 together with the excitation light and the fluorescence is shown in FIG. 2A and FIG. 2B. However, as described above, in the endoscope device 1, the weak external light (the visible light) incident on the distal end of the insertion unit 11 can be bundled as light similar to the excitation light. Accordingly, in the following description, weak visible light is not distinguished and is referred to as the “excitation light”.

The insertion unit 11 guides the excitation light irradiated by the light source 111 reflected from the test subject incident on the distal end and the fluorescence emitted by ICG through excitation by the excitation light to the excitation light cut filter 121. Here, a light intensity of the fluorescence incident on the excitation light cut filter 121 is weaker than a light intensity of the excitation light as shown in (a) of FIG. 2A. Thus, in the endoscope device 1, only the excitation light included in the incident light is attenuated by the excitation light cut filter 121. Thereby, the light emitted from the excitation light cut filter 121 is in a state in which the light intensity of the excitation light is weaker than the light intensity of the fluorescence as shown in (b) of FIG. 2A.

However, as shown in (b) of FIG. 2A, the light emitted from the excitation light cut filter 121 includes not only a wavelength band (component) of the fluorescence but also a wavelength band of the weak excitation light (which also includes the weak visible light) as a noise component. As shown in (c) of FIG. 1A, a range indicated by a dotted line in (b) of FIG. 2A is enlarged. As shown in (c) of FIG. 2A, the weak excitation light (which also includes the weak visible light), which cannot be attenuated by the excitation light cut filter 121, is included to the light emitted from the excitation light cut filter 121 as the noise component.

In the conventional endoscope device, photographing is performed with light in the state shown in (b) of FIG. 2A and (c) of FIG. 2A, that is, the fluorescence in a state in which the noise component is included. Thus, the image of the test subject generated through photographing by the conventional endoscope device contains a noise component in addition to a fluorescence component. Thus, in the conventional endoscope device, as shown in (c) of FIG. 2A, if the difference between the light intensity of the fluorescence component and the light intensity of the noise component is not large, that is, if the fluorescence is minute, it may be difficult to distinguish between the noise component and the fluorescence component, and it may be impossible to identify a light emitting portion with respect to the fluorescence in the test subject.

On the other hand, in the endoscope device 1, the fluorescence including the noise component as shown in (b) of FIG. 2A and (c) of FIG. 2A output from the excitation light cut filter 121 is split off by the half mirror 122. In the endoscope device 1, the imager 123 performs photographing with fluorescence in a state in which a noise component is included as in the conventional endoscope device. Also, in the endoscope device 1, the fluorescence cut filter 124 eliminates the fluorescence component from the fluorescence in a state in which the noise component is included, extracts only the noise component, and the imager 125 performs photographing with light of the noise component. That is, in the endoscope device 1, the imager 125 performs photographing with light of a wavelength band (component) of the weak excitation light (which also includes the weak visible light) that cannot be eliminated by the excitation light cut filter 121.

Then, in the endoscope device 1, the signal processing unit 21 generates an image of the fluorescence including the weak excitation light on the basis of a pixel signal obtained through photographing by the imager 123, and generates an image of only the weak excitation light on the basis of the pixel signal obtained through the photographing by the imager 125. Thereafter, in the endoscope device 1, an image of the test subject captured in a state of there being only fluorescence (an image of only the fluorescence component) is generated by subtracting the image of only the weak excitation light from the image of the fluorescence including the weak excitation light.

A process of the endoscope device 1 as described above is schematically shown in FIG. 2B. The hunger 123 performs photographing similar to that of the conventional endoscope device with the light of the wavelength band as shown in (a) of FIG. 2B, that is, the fluorescence including the noise component as shown in (b) of FIG. 2A and (c) of the FIG. 2A, and the imager 125 performs photographing with light in a wavelength band other than the wavelength band of the fluorescence as shown in (b) of FIG. 2B. Thereby, an image including a wavelength band (component) in which the weak excitation light (including the weak visible light) and the fluorescence are combined (an image of the fluorescence including the weak excitation light) is generated from the pixel signal output to the signal processing unit 21 by the imager 123. Also, an image including only the wavelength band (component) of weak excitation light (which also includes weak visible light) (an image of only the weak excitation light) is generated from the pixel signal output to the signal processing unit 21 by the imager 125.

The signal processing unit 21 generates an image obtained by subtracting the image generated on the basis of the pixel signal input from the imager 125 from the image generated on the basis of the pixel signal input boot the imager 123. That is, the signal processing unit 21 generates an image corresponding to the difference between the image of the fluorescence including the weak excitation light and the image of only the weak excitation light. Thereby, the signal processing unit 21 generates an image including light of the wavelength band as shown in (c) of FIG. 2B, that is, an image of only the wavelength band (component) of the fluorescence which does not include the noise component. Thereby, the endoscope device 1 generates an image captured in a state of there being only fluorescence.

According to the first embodiment, an infrared fluorescence observation device (the endoscope device 1) includes a light source (the light source 111) configured to irradiate visible light and excitation light including a longer was band than the visible light; an imaging unit on which the visible light, the excitation light, and fluorescence including a longer wavelength band than the excitation light are incident from a subject (the test subject) irradiated by the light source 111; and a signal processing unit (the signal processing unit 21) configured to process a signal obtained from the imaging unit, wherein the imaging unit generates a first image (the image of the fluorescence including weak excitation light) on the basis of second light (the fluorescence and the weak excitation light) obtained be attenuating a wavelength band including the excitation light from first light (the excitation light and the fluorescence) emitted from the test subject irradiated with at least the excitation light, and generates a second image (the image of only the weak excitation light) on the basis of third light (the weak excitation light) obtained by eliminating only the wavelength band of the fluorescence from the fluorescence and the weak excitation light, and wherein the signal processing unit 21 generates a third image (the image of only the fluorescence component) according to light of a wavelength band including the fluorescence using the image of the fluorescence including the weak excitation light and the image of only the weak excitation light.

Also, according to the first embodiment, the endoscope device 1 is configured so that the signal processing unit 21 generates the image of only the fluorescence component by subtracting the image of only the weak excitation light from the fluorescence image including the weak excitation light.

Also, according to the first embodiment, the endoscope device 1 is configured so that the imaging unit includes a first wavelength selection unit (the excitation light cut filter 121) configured is input excitation light and fluorescence and output fluorescence and weak excitation light; a half mirror (the half mirror 122) configured to divide the fluorescence and the weak excitation light into a first optical path (the optical path of the imager 123 side) and a second optical path (an optical path of the imager 125 side); a first imaging element (the imager 123) arranged in the optical path of the imager 123 side and configured to generate the image of the fluorescence including the weak excitation light on the basis of the fluorescence and the weak excitation light; a second wavelength selection unit (the fluorescence cut filter 124) arranged in the optical path of the imager 125 side and configured is input the fluorescence and the weak excitation light, eliminate a wavelength band including the fluorescence, and output the weak excitation light; and a second imaging element (the imager 125) arranged in the optical path of the imager 125 side and configured to generate the image of only the weak excitation light on the basis of the weak excitation light.

Also, according to the first embodiment, the infrared fluorescence observation device is an endoscope device (the endoscope device 1), and the endoscope device 1 includes a scope unit (the scope unit 10) including an insertion unit (the insertion unit 11) configured to be inserted into a body (the body of the test subject person in a state in which ICG has been administered in advance) and an operation unit (the operation unit 12) configured to operate the insertion unit 11; and an external processing unit (the external processing unit 20) connected to the scope unit 10, wherein the light source 111 and the imaging unit are arranged in the scope unit 10, and wherein the signal processing unit 21 is arranged in the external processing unit 20.

As described above, in the endoscope device 1 of the first embodiment, the excitation light component is attenuated from light in which the excitation light reflected from the test subject which is incident when irradiating the excitation light and the fluorescence emitted through excitation of ICG by the excitation light are combined. Then, in the endoscope device 1 of the first embodiment, an image of the test subject photographed with only the fluorescence is generated by performing photographing with light in which the excitation light component is attenuated and photographing with light from which the fluorescence is further eliminated and taking a difference between images generated on the basis of the pixel signals obtained in each the photographing. Thereby, in the endoscope device 1 according to the first embodiment, it is possible to obtain an image of the test subject including only a fluorescence component by a method which is easier than including a mechanism for further eliminating a weak excitation light component which cannot be eliminated (which also includes the weak visible light) in the conventional endoscope device.

That is, in the endoscope device 1 according to the first embodiment, a mechanism for easily obtaining an image in which the excitation light (which also includes the visible light) having a strong light intensity is eliminated by performing calculation using an image obtained by eliminating the fluorescence which is easily eliminated because the light intensity is originally weak is constructed without constructing a mechanism for eliminating the excitation light (which also includes the visible light) having an originally strong light intensity. Moreover, calculation for obtaining an image including only the fluorescence component in the endoscope device 1 of the first embodiment is only a simple calculation, that is, only a difference calculation. Thereby, in the endoscope device 1 of the first embodiment, even if the fluorescence is minute, the excitation light and the fluorescence are separated with high accuracy and an image of the test subject containing only the fluorescence component can be obtained.

Also, in the endoscope device 1 of the first embodiment, the calculation for obtaining the image including only the component of fluorescence is not limited to the difference calculation as described above. For example, after one or both of a pixel included in the image of the fluorescence including the weak excitation light according to the pixel signal from the imager 123 and a pixel included in the image of only the weak excitation light according to the pixel signal from the imager 125 are multiplied by a predetermined pixel value, a difference calculation between pixel values of pixels arranged at the same position in the images may be performed. Thereby, for example, even if the sensitivity of the light of the imager 123 is different from the sensitivity of the light of the imager 125 or the ratio of splitting the incident light of the half mirror 122 is not 1:1, an image of the fluorescence including the weak excitation light and an image of only the weak excitation light can be made to have similar luminance levels. Thereby, it is possible to make an image subjected to the difference calculation, that is, an image of the test subject, with a more accurate luminance level.

### Second Embodiment

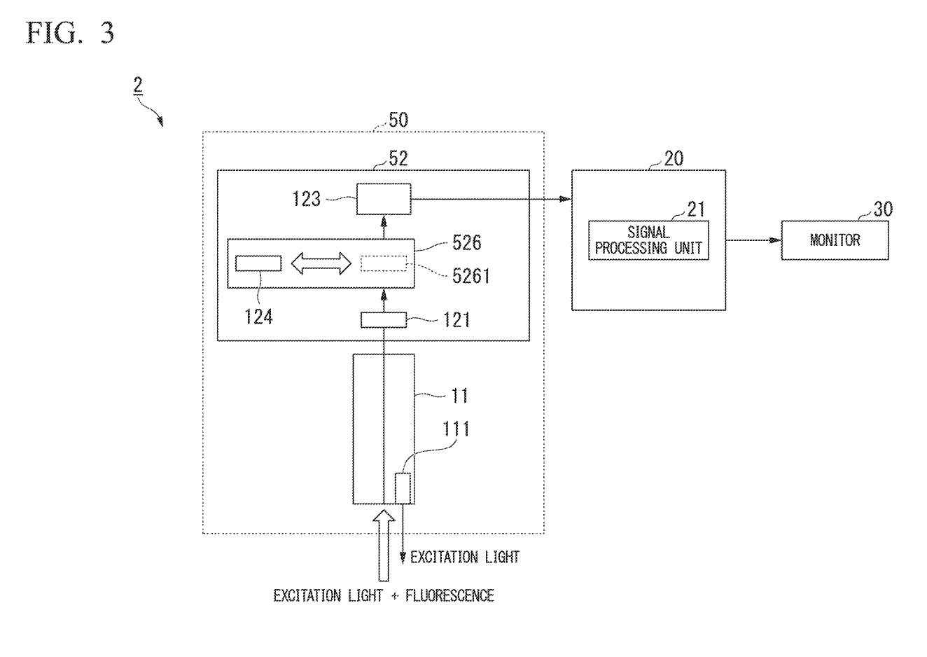

Next, the second embodiment of the present invention will be described. In the second embodiment, as in the first embodiment, a case in which the infrared fluorescence observation device of the present invention is configured as an endoscope device will be described. FIG. 3 is a diagram showing a schematic configuration of the endoscope device according to the second embodiment of the present invention.

Similar to the endoscope device 1 of the first embodiment, an endoscope device 2 of the second embodiment is also a rigid endoscope for laparoscopic surgery and is used fir the test subject person in a state in which a fluorescent drug such as ICG has been administered into his/her body in advance. Similar to the endoscope device 1 of the first embodiment, the endoscope device 2 also has a function of photographing the test subject with visible light and a function of photographing the test subject with fluorescence emitted by excitation of administered ICG through irradiation of excitation light such as near infrared light.

In FIG. 3, the endoscope device 2 includes a scope unit 50, an external processing unit 20, and a monitor 30. In the endoscope device 2, the scope unit 50 includes an insertion unit 11 and an operation unit 52. The configuration of the imaging unit of the endoscope device 2 is different from that of the imaging unit included in the endoscope device 1 of the first embodiment. The endoscope device 2 includes constituent elements similar to those provided in the endoscope device 1 of the first embodiment. Accordingly, in the following description, the same reference signs are assigned to constituent elements of the endoscope device 2 similar to those included in the endoscope device 1 of the first embodiment shown in FIG. 1, a detailed description of these constituent components will be omitted, and only differences from the endoscope device 1 of the first embodiment will be described with respect to the endoscope device 2.

In the scope unit 50, the operation unit 52 is configured to include an imaging unit having an excitation light cut filter 121, a filter switching unit 526, and an imager 123, which are constituent elements of the infrared fluorescence observation device of the present invention. In the endoscope device 2 of the second embodiment, the infrared fluorescence observation device of the present invention includes a light source 111, the imaging unit (the excitation light cut filter 121, the filter switching unit 526, and the imager 123), and a signal processing unit 21.

The insertion unit 11 provided in the scope unit 50 guides the incident excitation light and fluorescence to the operation unit 52 provided in the scope unit 50.

Similar to the imaging unit provided in the operation unit 12 provided in the scope unit 10 of the endoscope device 1 according to the first embodiment, the imaging unit provided in the operation unit 52 outputs a pixel signal obtained through photographing by the imager 123 to the external processing unit 20.

The excitation light cut filter 121 emits light (fluorescence) attenuated by reflecting or absorbing the incident excitation light and only the excitation light included in the fluorescence to the filter switching unit 526. Also, the light emitted from the excitation light cut filter 121 contains weak excitation light as in the endoscope device 1 of the first embodiment.

The filter switching unit 526 switches between emitting the light incident from the excitation light cut filter 121, to the imager 123 without passing through the optical filter and emitting the light to the imager 123 through the optical filter. In FIG. 3, a light transmission window 5261 indicated by a dotted lane in the filter switching unit 526 is an opening for emitting the fluorescence including the weak excitation light incident from the excitation light cut filter 121 to the imager 123 as it is.

The filter switching unit 526 switches between a mode in which light is emitted without passing through the optical filter and a mode in which light passing through the optical filter, is emitted for each frame captured by the imager 123. That is, the filter switching unit 526 switches the mode in which light is emitted to one of the modes in synchronization with the frame captured by the imager 123.

More specifically, the filter switching unit 526 performs switching to the mode in which the light is emitted without passing through the optical filter during a frame in which the imager 123 acquires a pixel signal according to the fluorescence and the weak excitation light. Thereby, the fluorescence containing the weak excitation light incident from the excitation light cut filter 121 is emitted to the hunger 123 through the light transmission window 5261 as it is.

Also, the filter switching unit 526 performs switching to the mode in which the light passing through the optical filter is emitted during a frame in which the imager 123 acquires a pixel signal according to the weak excitation light. Thereby, the fluorescence including the weak excitation light incident from the excitation light cut filter 121 is emitted to the imager 123 through the fluorescence cut filter 124. That is, the weak excitation light from which only the fluorescence component is eliminated is emitted to the imager 123.

In the present invention, the configuration in which the filter switching unit 526 switches the light emitted to the hunger 123, that is, the configuration in which the fluorescence cut filter 124 and the light transmission window 5261 are switched according to each mode is not particularly limited. For example, a configuration in which the filter switching unit 526 is constituted off disk on which the fluorescence cut filter 124 and the light transmission window 5261 are arranged, and switches the light emitted to the imager 123 by rotating the disk in synchronization with the frame captured by the imager 123 may be adopted. Also, for example, a configuration in which the filter switching unit 526 is constituted of a rectangular base (a plate) on which the fluorescence cut filter 124 and the light transmission window 5261 are arranged, and switches the light emitted to the hunger 123 by sliding the base to slide in synchronization with a frame captured by the hunger 123 may be adopted.

Also, in the present invention, a configuration by which a timing at which the filter switching unit 526 switches the light emitted to the imager 123, that is, a timing for switching to each mode, is controlled is not particularly limited. For example, a configuration in which a drive circuit (not shown) for driving the imager 123 or an imaging control unit (not shown) for controlling photographing in the endoscope device 2 outputs a control signal indicating a type of light in which a pixel signal is acquired in accordance with the timing of each frame captured by the hunger 123, and the filter switching unit 526 may switch the light emitted to the imager 123 in accordance with this control signal may be adopted.

The imager 123 performs photographing with the fluorescence including the weak excitation light emitted from the filter switching unit 526, or the weak excitation light, and sequentially outputs pixel signals obtained by the photographing to the external processing unit 20. That is, the imager 123 alternately outputs a pixel signal of a frame captured with the fluorescence including the weak excitation light, and a pixel signal of a frame captured with the weak excitation light to the external processing unit 20.

The signal processing unit 21 provided in the external processing unit 20 generates an image of the test subject formed by only a pixel signal according to the fluorescence on the basis of each pixel signal sequentially input from the operation unit 52 provided in the scope unit 50, and outputs the generated image of the test subject to the monitor 30 for display. That is, the signal processing unit 21 generates an image formed by only the pixel signal according to the fluorescence (an image of only the fluorescence component) by subtracting the image of only the weak excitation light generated on the basis of the pixel signal according to the weak excitation light input from the same imager 123 from the image of the fluorescence including the weak excitation light generated on the basis of the pixel signal according to the fluorescence including the weak excitation light input from the imager 123, and outputs the generated image to the monitor 30.

With such a configuration, similar to the endoscope device 1 of the first embodiment, the endoscope device 2 excites ICG administered into the test subject person with the excitation light, and presents an image of the test subject according to the fluorescence emitted by the excited ICG to the examiner.

Also, a method of generating an image of only the fluorescence component in the signal processing unit 21 provided in the external processing unit 20 of the endoscope device 2 is similar to the image generation method of the signal processing unit 21 in the endoscope device 1 of the first embodiment shown in FIG. 2A and FIG. 2B, except that a timing at which the pixel signal is input to the signal processing unit 21 is different. That is, although it is also conceivable that pixel signals are simultaneously input from the imager 123 and the imager 125 to the signal processing unit 21 in the endoscope device 1 of the first embodiment, there is a difference in that the pixel signals obtained through photographing in the modes are sequentially input from the imager 123 to the signal processing unit 21, in the endoscope device 2. Accordingly, a detailed description of a method of generating an image captured in a state of only fluorescence in the endoscope device 2 is omitted.

According to the second embodiment, the infrared fluorescence observation device (the endoscope device 2) includes a switching unit (the filter switching unit 526) configured to sequentially switch between a first mode (the mode in which the light is emitted without passing through the optical filter) and a second mode (the mode in which the light passing through the optical filter is emitted), wherein the light source (the light source 111) irradiates at least the excitation light in the mode in which the light is emitted without passing through the optical filter and the mode in which the light passing through the optical filter is emitted, and wherein the imaging unit includes a first wavelength selection unit (the excitation light cut filter 121) configured is input the first light (the excitation light and the fluorescence), attenuate a wavelength band including the excitation light, and output the second light (the fluorescence and the weak excitation light) to a predetermined optical path (the optical path of the imager 123); a second wavelength selection unit (the fluorescence cut filter 124) arranged to be retracted from the optical path of the imager 123 in the mode in which the light is emitted without passing through the optical filter, arranged in the optical path of the imager 123 in the mode in which the light passing through the optical filter is emitted, and configured is input the fluorescence and the weak excitation light, eliminate a wavelength band including the fluorescence, and output the third light (the weak excitation light); and an imaging element (the imager 123) arranged in the optical path of the imager 123 and configured to generate the first image (the image of the fluorescence including the weak excitation light) on the basis of the fluorescence and the weak excitation light in the mode in which the light is emitted without passing through the optical filter and generate the second image (the image of only the weak excitation light) on the basis of the weak excitation light in the mode in which the light passing through the optical filter is emitted.

As described above, in the endoscope device 2 of the second embodiment, as in the endoscope device 1 of the first embodiment, the excitation light component is attenuated from light in which the excitation light reflected from the test subject which is incident when irradiating the excitation light and the fluorescence emitted through excitation of ICG by the excitation light are combined. Then, in the endoscope device 2 of the second embodiment, photographing with light in which the excitation light component is attenuated (photographing in the mode in which the light is emitted without passing through the fluorescence cut filter 124) and photographing with light obtained by further eliminating fluorescence (photographing in the mode in which the light passing through the fluorescence cut filter 124 is emitted) are alternately performed for each frame, and a difference between images generated on the basis of pixel signals obtained in each the photographing is taken, so that an image of the test subject captured with only the fluorescence is generated. Thereby, also in the endoscope device 2 of the second embodiment, it is possible to obtain an image of the test subject including only the fluorescence component in a method which is easier than in the conventional endoscope device. Thereby, in the endoscope device 2 of the second embodiment, as in the endoscope device 1 of the first embodiment, even if the fluorescence is minute, the excitation light and the fluorescence are separated with high accuracy and an image of the test subject containing only the fluorescence component can be obtained.

Moreover, in the endoscope device 2 of the second embodiment, the photographing in the mode in which the light is emitted without passing through the fluorescence cut filter 124 and the photographing in the mode in which the light passing through the fluorescence cut filter 124 is emitted are performed by the same single imager 123. Thus, in the endoscope device 2 of the second embodiment, even if the calculation for obtaining the image including only the fluorescence component is only the above-described difference calculation, an image of the test subject can be obtained with an accurate luminance level. Also, because the sane single imager 123 performs photographing in each mode in the endoscope device 2 of the second embodiment, the angle of view of the photographing is not shifted. That is, the position of the test subject photographed in each mode is not shifted.

### Third Embodiment

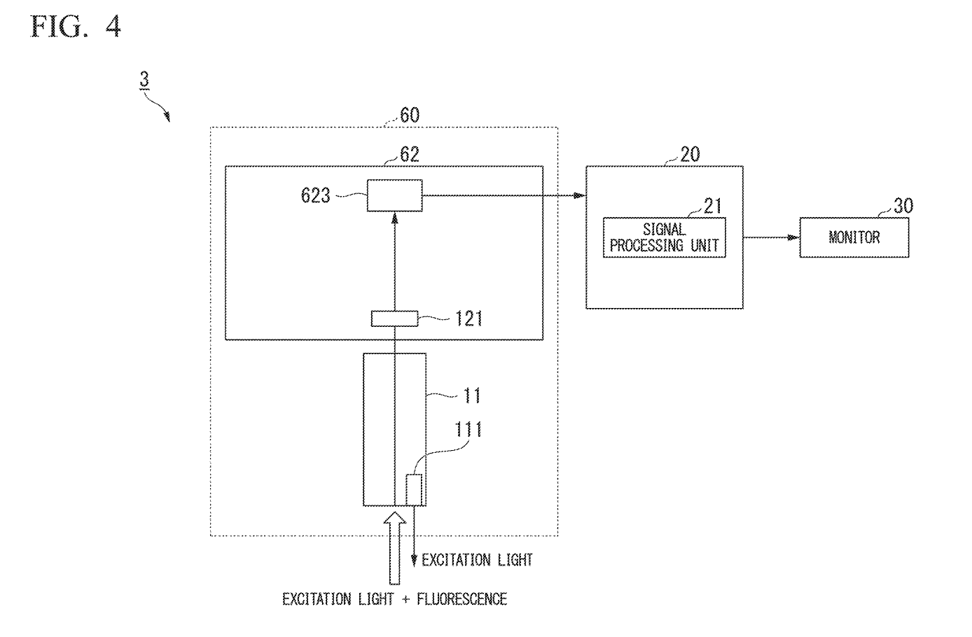

Next, the third embodiment of the present invention will be described. Also in the third embodiment, as in the first embodiment and the second embodiment, a case in which the infrared fluorescence observation device of the present invention is configured as an endoscope device will be described. FIG. 4 is a diagram showing a schematic configuration of the endoscope device according to the third embodiment of the present invention.

Similar to the endoscope device 1 of the first embodiment and the endoscope device 2 of the second embodiment, an endoscope device 3 of the third embodiment is also a rigid endoscope for laparoscopic surgery and is used for a test subject person in a state in which a fluorescent drug such as ICG has been administered into his/her body in advance. Also, similar to the endoscope device 1 according to the first embodiment and the endoscope device 2 of the second embodiment, the endoscope device 3 also has a function of photographing the test subject with visible light and a function of photographing the test subject with fluorescence emitted by excitation of administered ICG through irradiation of excitation light such as near infrared light.

In FIG. 4, the endoscope device 3 includes a scope unit 60, an external processing unit 20, and a monitor 30. In the endoscope device 3, the scope unit 60 includes an insertion unit 11 and an operation unit 62. The configuration of the imaging unit of the endoscope device 3 is different from the configuration of the imaging unit included in the endoscope device 1 of the first embodiment or the endoscope device 2 of the second embodiment. The endoscope device 3 includes constituent elements similar to those provided in the endoscope device 1 of the first embodiment and the endoscope device 2 of the second embodiment. Accordingly, in the following description, among the constituent elements of the endoscope device 3, constituent elements similar to those of the endoscope device 1 of the first embodiment shown in FIG. 1 or the endoscope device 2 of the second embodiment shown in FIG. 3 are denoted by the same reference signs and a detailed description of these constituent elements will be omitted, and only differences from the endoscope device 1 of the first embodiment and the endoscope device 2 of the second embodiment will be described with respect to the endoscope device 3.

In the scope unit 60, the operation unit 62 is configured to include an imaging unit having an excitation light cut filter 121, and an imager 623, which are constituent elements of the infrared fluorescence observation device of the present invention. In the endoscope device 3 of the third embodiment, the infrared fluorescence observation device of the present invention includes a light source 111, the imaging unit the excitation light cut filter 121 and the imager 623), and a signal processing unit 21.

The insertion unit 11 provided in the scope unit 60 guides the incident excitation light and fluorescence to the operation unit 62 provided in the scope unit 60.

Similar to the imaging unit provided in the operation unit 12 provided in the scope unit 10 of the endoscope device 1 according to the first embodiment and the imaging unit provided in the operation unit 62 provided in the scope unit 50 of the endoscope device 2 according to the second embodiment, the imaging unit provided in the operation unit 62 outputs a pixel signal obtained through photographing by the imager 623 to the external processing unit 20.

The excitation light cut filter 121 emits light (fluorescence) attenuated by reflecting or absorbing the incident excitation light and only the excitation light included in the fluorescence to the imager 623. Also, the light emitted from the excitation light cut filter 121 contains weak excitation light as in the endoscope device 1 of the first embodiment and the endoscope device 2 of the second embodiment.

Similar to the imager 123 of the endoscope device 1 of the first embodiment and the endoscope device 2 of the second embodiment, the imager 623 is an imaging element that exposes (detects) the incident light and outputs a pixel signal obtained by photoelectrically converting the exposed light. Also, in the imager 623, a plurality of pixels for exposing (detecting) light having different wavelengths are arranged.

More specifically, in the imager 623, a plurality of pixels for exposing fluorescence including weak excitation light and a plurality of pixels for exposing only the weak excitation light are arranged on a surface of a side on which light is incident. The pixels for exposing only the weak excitation light are pixels to which the fluorescence cut filter similar to the fluorescence cut filter 124 provided in the endoscope device 1 of the first embodiment and the endoscope device 2 of the second embodiment is attached as an on chip color filter. On the other hand, a pixel for exposing fluorescence containing weak excitation light is a pixel to which no fluorescence cut filter is attached. In other words, pixels for exposing the fluorescence including the weak excitation light are similar to pixels arranged in the imager 123 provided in the endoscope device 1 of the first embodiment and the endoscope device 2 of the second embodiment.

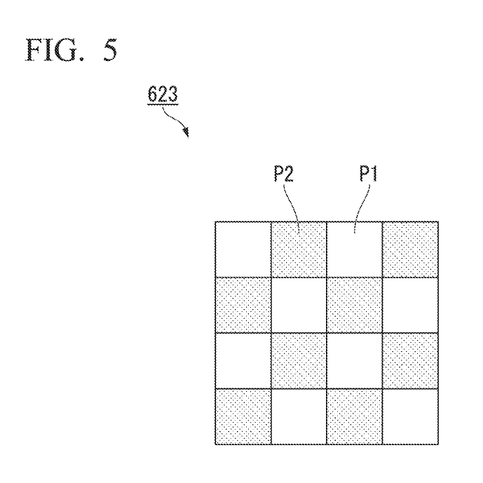

FIG. 5 is a diagram showing an example of the arrangement of pixels in the imager 623 provided in the endoscope device 3 of the third embodiment of the present invention. In FIG. 5, the imager 623 in which a pixel P1 (hereinafter referred to as an “unfiltered pixel P1”) for exposing the fluorescence including the weak excitation light and a pixel P2 (hereinafter referred to as a “filtered pixel P2”) for exposing only the weak excitation light are alternately arranged in the horizontal direction and the vertical direction, that is, arranged in a so-called checkered pattern, is shown.

The imager 623 performs photographing with the fluorescence including the weak excitation light emitted from the excitation light cut filter 121 and outputs a pixel signal obtained by the unfiltered pixel P1 and a pixel signal obtained by the filtered pixel P2 to the external processing unit 20.

Also, the arrangement of the unfiltered pixels P1 and the filtered pixels P2 in the imager 623, and the configuration of the imager 623 are not limited to the above-described arrangement and configuration.

The signal processing unit 21 provided in the external processing unit 20 generates an image of the test subject farmed by only the pixel signal according to the fluorescence on the basis of each pixel signal input from the operation unit 62 provided in the scope unit 60. More specifically, the signal processing unit 21 generates an image formed by only the pixel signal according to the fluorescence (an image of only the fluorescence component) by subtracting an image of only the weak excitation light generated on the basis of the pixel signals of the filtered pixels P2 from an image of the fluorescence including the weak excitation light generated on the basis of the pixel signals of the unfiltered pixels P1 input from the imager 623 provided in the operation unit 62.

At this time, when an image according to pixel signals is generated, the signal processing unit 21 calculates a difference between pixel values of pixels arranged at the same position in images after the image including pixel signals of all pixels is generated by interpolating a pixel signal at a position where a pixel different from the pixel from which the input pixel signal is obtained is arranged, that is, a pixel signal of a lost pixel. More specifically, if generating a fluorescence image including the weak excitation light from the pixel signal of an unfiltered pixel P1, the pixel signal of an unfiltered pixel P1 at a position where a filtered pixel P2 is arranged is interpolated on the basis of a pixel signal of a surrounding unfiltered pixel P1. On the other hand, if generating an image of only weak excitation light from the pixel signal of a filtered pixel P2, the pixel signal of a filtered pixel P2 at a position where an unfiltered pixel P1 is arranged is interpolated on the basis of a pixel signal of a surrounding filtered pixel P2.

Also, in the present invention, a method in which the signal processing unit 21 interpolates pixel signals of pixels (a pixel interpolation operation method) is not particularly limited. For example, the pixel signal interpolation method may be a bilinear method or a bicubic method. Also, for example, the pixel signal interpolation method may be another method such as a nearest neighbor method.

The signal processing unit 21 outputs the image of the test subject of only the fluorescence component generated by taking the difference between the images generated by interpolating the pixel signals of each the pixels to the monitor 30 for display.

With such a configuration, similar to the endoscope device 1 of the first embodiment and the endoscope device 2 of the second embodiment, the endoscope device 3 excites ICG administered into the test subject person with the excitation light, and presents an image of the test subject according to the fluorescence emitted by the excited ICG to an examiner.

Also, as described above, a method of generating an image of only the fluorescence component in the signal processing unit 21 provided in the external processing unit 20 of the endoscope device 3 is similar to the image generation method of the signal processing unit 21 in the endoscope device 1 of the first embodiment shown in FIG. 2A and FIG. 2B, except that the signal processing unit 21 performs a process of interpolating a pixel included in each image on the basis of the input pixel signal. Accordingly, a detailed description of a method of generating an image captured in a state of only fluorescence in the endoscope device 3 will be omitted.

According to the third embodiment, the infrared fluorescence observation device (the endoscope device 3) is configured such that the imaging unit includes a first wavelength selection unit (the excitation light cut filter 121) configured is input the first light (the excitation light and the fluorescence), attenuate a wavelength band including the excitation light, and output the second light (the fluorescence and the weak excitation light); and an imaging element (the imager 623) configured is input the fluorescence and the weak excitation light, and wherein the imager 623 includes a plurality of first pixels (the unfiltered pixels P1) periodically arranged on a predetermined surface (the surface of the side on which the light is incident), having sensitivity to the fluorescence and the weak excitation light, and constituting the first image (the image of the fluorescence including the weak excitation light); and a plurality of second pixels (the filtered pixels P2) periodically arranged on the surface of the side on which the light is incident, each including a filter (the fluorescence cut filter: on chip color filter) for obtaining the third light (the weak excitation light) by eliminating a wavelength band including the fluorescence from the fluorescence and the weak excitation light, having sensitivity to the weak excitation light, and constituting the second image (the image of only the weak excitation light).

Also, according to the third embodiment, the endoscope device 3 is configured such that the unfiltered pixels P1 and the filtered pixels P2 are alternately arranged in a horizontal direction and a vertical direction.

As described above, in the endoscope device 3 of the third embodiment, as in the endoscope device 1 of the first embodiment and the endoscope device 2 of the second embodiment, the excitation light component is attenuated from light in which the excitation light reflected from the test subject which is incident when irradiating the excitation light and the fluorescence emitted through excitation of ICG by the excitation light are combined. Then, in the endoscope device 3 of the third embodiment, the imager 623 simultaneously acquires a pixel signal according to light obtained by attenuating the excitation light component and a pixel signal according to light obtained by further eliminating the fluorescence, and generates an image according to in each the acquired pixel signals. Thereafter, in the endoscope device 3 of the third embodiment, as in the endoscope device 1 of the first embodiment and the endoscope device 2 of the second embodiment, an image of the test subject captured with only the fluorescence is generated by taking a difference between images. Thereby, also in the endoscope device 3 of the third embodiment, it is possible to obtain an image of the test subject including only the fluorescence component in a method Which is easier than in the conventional endoscope device. Thereby, in the endoscope device 3 of the third embodiment, as in the endoscope device 1 of the first embodiment and the endoscope device 2 of the second embodiment, even if the fluorescence is minute, the excitation light and the fluorescence are separated with high accuracy and an image of the test subject containing only the fluorescence component can be obtained.

Moreover, in the endoscope device 3 of the third embodiment, acquisition of a pixel signal according to light obtained by attenuating the excitation light component and acquisition of a pixel signal according to light obtained by further eliminating the fluorescence are efficiently performed by the single imager 623, and each of the pixel signals are output at the same time. At this time, in the endoscope device 3 of the third embodiment, photographing with each of the light is performed using the unfiltered pixels P1 and the filtered pixels P2 provided in the imager 623, without switching between the light obtained by attenuating the excitation light component and the light obtained by further eliminating the fluorescence by the filter switching unit 526 as in the endoscope device 2 of the second embodiment. Thus, in the endoscope device 3 of the third embodiment, the imaging unit provided in the operation unit 62 can be made to be smaller than the imaging unit provided in the operation unit 52 of the endoscope device 2 in the second embodiment.

As described above, according to each embodiment of the present invention, the excitation light component is first attenuated, from light in which the excitation light reflected from the test subject which is incident when irradiating the excitation light and the fluorescence emitted through excitation of a fluorescent drug such as ICG by the excitation light are combined. Also, in each embodiment of the present invention, the fluorescence is further eliminated from the light obtained by attenuating the excitation light component. Then, in each embodiment of the present invention, an image of the test subject captured with only the fluorescence is generated by performing photographing with the light obtained by attenuating the excitation light component, and photographing with the light obtained by further eliminating the fluorescence, and taking a difference between images generated on the basis of pixel signals obtained in the photographing. Thereby, in each embodiment of the present invention, it is possible to separate the excitation light and the fluorescence with a high accuracy in a method which is easier than in the conventional technology. Thereby, in each embodiment of the present invention, even if the fluorescence emitted from a fluorescent drug is minute, an image of the test subject containing only the fluorescence component can be obtained.

In each embodiment of the present invention, the imaging unit constituting the infrared fluorescence observation device of the present invention is provided (arranged) in the operation unit constituting the scope unit of the endoscope device. However, the position where the imaging unit is arranged is not limited to the position shown in each embodiment. For example, the imaging unit constituting the infrared fluorescence observation device can be disposed at the distal end of the insertion unit constituting the scope unit of the endoscope device. In this case, by making the insertion unit of the endoscope device flexible, it is also possible to have a function of bending the insertion unit or the distal end of the insertion unit, for example, according to the operation of the operation unit by the examiner.

Also, in each embodiment of the present invention, a case in which the infrared fluorescence observation device of the present invention is configured as an endoscope device has been described. However, the infrared fluorescence observation device of the present invention is not limited to a configuration serving as the endoscope device shown in each embodiment. For example, the infrared fluorescence observation device of the present invention may be configured as a microscope device. In this case, each component of the infrared fluorescence observation device of the present invention is arranged at an appropriate position in the microscope device.

According to this, the infrared fluorescence observation device is configured as an infrared fluorescence observation device which is the microscope device.

While preferred embodiments of the present invention have been described and shown above, the invention is not limited to the embodiments and modified examples thereof. Within a range not departing from the gist or spirit of the present invention additions, omissions, substitutions, and other modifications to the configuration can be made.

Further, the present invention is not to be considered as being limited by the foregoing description, and is limited only by the scope of the appended claims.

What is claimed is:

1. An infrared fluorescence observation device comprising:
a light source configured to irradiate visible light and excitation light including a longer wavelength band than the visible light;
an imaging unit on which the visible light, the excitation light, and fluorescence including a longer wavelength band than the excitation light are incident from a subject irradiated by the light source; and
a signal processing unit configured to process a signal obtained from the imaging unit,
wherein the imaging unit includes
a first wavelength selection unit configured is input first light from the subject irradiated with at least the excitation light and output second light obtained by attenuating a wavelength band including the excitation light from the first light;
a half mirror configured to divide the second light into a first optical path and a second optical path;
a first imaging element arranged in the first optical path and configured to generate a first image on the basis of the second light;
a second wavelength selection unit arranged in the second optical path, to which the second light is input, and from which third light obtained by eliminating only a wavelength band including the fluorescence from the second light is output; and
a second imaging element arranged in the second optical path and configured to generate a second image on the basis of the third light, and
wherein the signal processing unit generates a third image according to light of a wavelength band including the fluorescence using the first image and the second image.

2. The infrared fluorescence observation device according to claim 1,
wherein the signal processing unit generates the third image by subtracting the second image from the first image.

3. The infrared fluorescence observation device according to claim 1,
wherein the infrared fluorescence observation device is an endoscope device,
wherein the endoscope device includes:
a scope unit having an insertion unit configured to be inserted into a body and an operation unit configured to operate the insertion unit; and
an external processing unit connected to the scope unit,
wherein the light source and the imaging unit are arranged in the scope unit, and
wherein the signal processing unit is arranged in the external processing unit.

4. The infrared fluorescence observation device according to claim 1,
wherein the infrared fluorescence observation device is a microscope device.
